(19) United States
(12) Patent Application Publication
(10) Pub. No.: US 2025/0011169 A1
(43) Pub. Date: Jan. 9, 2025

(54) APPARATUS FOR MANUFACTURING HYDROGEN PEROXIDE WATER BY USING ELECTROSTATIC SPRAYING

(71) Applicant: KOREA INSTITUTE OF ENERGY RESEARCH, Daejeon (KR)

(72) Inventors: Jong Won CHOI, Daejeon (KR); Hak Geun JEONG, Daejeon (KR); Young Chan CHOI, Daejeon (KR); Gil Bong LEE, Daejeon (KR); Young Joo LEE, Sejong-si (KR); Kang San LEE, Sejong-si (KR); Jun Su PARK, Sejong-si (KR)

(73) Assignee: KOREA INSTITUTE OF ENERGY RESEARCH, Daejeon (KR)

(21) Appl. No.: 18/709,332

(22) PCT Filed: Nov. 12, 2021
(86) PCT No.: PCT/KR2021/016567

§ 371 Date: May 23, 2024

(30) Foreign Application Priority Data
Nov. 12, 2021 (KR) 10-2021-0155842

(51) Int. Cl. C01B 15/029; B05B 5/03

(52) U.S. Cl. CPC C01B 15/0295; B05B 5/03

(57) ABSTRACT

An apparatus for manufacturing hydrogen peroxide water by using electrostatic spraying is disclosed. An apparatus for manufacturing hydrogen peroxide water by using electrostatic spraying according to a first embodiment of the present disclosure comprises: a nozzle unit for spraying reaction water; a ground unit disposed opposite to the nozzle unit; and a power supply unit for applying a high voltage between the nozzle unit and the ground unit, wherein the reaction water is formed into fine droplets with a particle size of 20 μm or less in the nozzle unit and is electrostatically sprayed, and ionized hydrogen ions (H⁺) and hydroxide ions (OH⁻) react with each other again to form hydrogen peroxide.

### TECHNICAL FIELD

The present disclosure relates to an apparatus for preparing a hydrogen peroxide solution using electrostatic spraying.

### BACKGROUND ART

Hydrogen peroxide (H₂O₂) is used as bleaching agents for pulp and fibers, disinfectants, sterilizers, semiconductor cleaning solutions, oxidants in water treatment processes and eco-friendly oxidants (propylene oxide synthesis) in chemical reactions.

As of 2009, 2,200,000 tons of hydrogen peroxide is produced per year and with the growing demand for propylene oxide, the demand for hydrogen peroxide is expected to increase.

Currently, hydrogen peroxide is produced through continuous oxidation and hydrogenation of anthraquinone-based compounds, and in this instance, a large amount of organic solvents are used, producing waste which adversely affects humans. Additionally, the production of hydrogen peroxide requires high energy consumption due to a multistep continuous process and purification and concentration after production.

Korean Patent Publication No. 10-2002-0032225 discloses a direct production process in which hydrogen peroxide is synthesized by direct reaction between hydrogen and oxygen, and the direct production process produces water as a by-product of the reaction and uses a small amount of organic solvents, so it has been studied as an alternative to the existing process.

The direct production process is simple and straightforward and can produce hydrogen peroxide on-site where hydrogen peroxide is needed, thereby significantly reducing explosion risks during storage and transportation of hydrogen peroxide. However, hydrogen peroxide is produced by injecting reducing agents and oxygen gas at the same time in the presence of catalysts using quinone compounds, so there are disadvantages in terms of the use of expensive catalysts and reactivity.

Additionally, in Korean Patent Publication No. 10-2020-0116734, direct hydrogen peroxide production reaction includes a reaction between hydrogen and oxygen to produce hydrogen peroxide as well as a side reaction to produce water. Because the side reaction is also a spontaneous reaction, studies are being conducted to increase hydrogen peroxide selectivity using catalysts.

The catalysts for direct hydrogen peroxide production mainly include palladium (Pd) or Pd alloys (Pd—Au, Pd—Pt), and in the case of palladium catalysts, many studies are being conducted to increase hydrogen peroxide selectivity by adding acids and halogen anions to solvents. However, there are disadvantages such as expensive catalysts, complex process and high operating costs, and large-scale equipment is required.

Additionally, Korean Patent Publication No. 10-2019-0055955 discloses evaporation of ionic water using electrostatic spraying to desalinate ionic water such as sea water by condensation of the remaining ionic water after evaporation at the rear end, but does not disclose hydrogen peroxide production through reaction.

Hydrogen peroxide (H₂O₂) is used in a wide range of industrial applications including disinfection and bleaching. However, high concentration hydrogen peroxide (H₂O₂) chemically produced and supplied has a risk of human injury, and thus it is recommended to produce on-site if necessary.

Additionally, recently, with the global spread of infectious diseases such as SARS, MERS and COVID19, there is increasing demand for sanitizing solutions to sanitize a variety of physical spaces. To prepare sanitizing solutions, a variety of technologies such as electrochemical devices, metal catalysts and plasma are used, but expensive metal catalysts and high energy consumption are required.

Accordingly, there is a need for studies on effective production of hydrogen peroxide without expensive metal catalysts and high energy consumption.

### PATENT LITERATURE

[Patent Literature 1] Korean Patent Publication No. 10-2002-0032225
[Patent Literature 2] Korean Patent Publication No. 10-2020-0116734
[Patent Literature 3] Korean Patent Publication No. 10-2019-0055955

### DISCLOSURE

### Technical Problem

The present disclosure is directed to providing an apparatus for preparing a hydrogen peroxide solution using electrostatic spraying without expensive metal catalysts and high energy consumption by atomizing a reacting solution into fine droplets through electrostatic spraying and reacting hydrogen ions with hydroxide ions again to produce hydrogen peroxide.

### Technical Solution

According to an aspect of the present disclosure, there is provided an apparatus for preparing a hydrogen peroxide solution using electrostatic spraying, including: a nozzle unit configured to spray a reacting solution; a ground unit disposed opposite the nozzle unit; and a power supply unit configured to apply a high voltage between the nozzle unit and the ground unit, wherein the reacting solution is atomized into fine droplets having a droplet size of 20 μm or less and electrostatic sprayed from the nozzle unit and hydrogen ions (H⁺) react with hydroxide ions (OH⁻) again to produce hydrogen peroxide.

The nozzle unit may include a nozzle body configured to supply the reacting solution; and a nozzle tip connected to the nozzle body, disposed opposite the ground unit and having an outlet through which the reacting solution is sprayed, and an average water droplet size of the reacting solution coming out of the outlet may be from 10 nm to 200 μm.

The nozzle tip may be rotatably connected to the nozzle body such that the nozzle tip is disposed in a coaxial direction or inclined with respect to the nozzle body.

The apparatus may further include a storage tank disposed below the nozzle unit and the ground unit to store the reacting solution including hydrogen peroxide produced through electrostatic spraying; and a filter unit connected in communication with the storage tank and configured to remove chlorine from the reacting solution including hydrogen peroxide.

According to another aspect of the present disclosure, there is provided an apparatus for preparing a hydrogen peroxide solution using electrostatic spraying, including: a reacting solution storage tank configured to store a reacting solution; a needle unit disposed below the reacting solution storage tank, and configured to spray the reacting solution fed from the reacting solution storage tank; a ground unit located below the needle unit and disposed opposite the needle unit; a storage tank disposed below the ground unit and configured to store the reacting solution including hydrogen peroxide; and a power supply unit configured to apply a high voltage between the nozzle unit and the ground unit, wherein the reacting solution is atomized into fine droplets having a droplet size of 20 μm or less and electrostatic sprayed from the needle unit and hydrogen ions (H⁺) react with hydroxide ions (OH⁻) again to produce hydrogen peroxide.

The ground unit may include a mesh-type ground plate disposed below the needle unit; and a cone-shaped anti-leak plate coupled to an edge of the ground plate and inclined upward.

The needle unit may include a conductive plate coupled to a lower surface of the reacting solution storage tank; and at least one needle coupled to a lower surface of the conductive plate and disposed opposite the ground unit to spray the reacting solution.

When a concentration (ppm) of hydrogen peroxide stored in the storage tank is less than a preset value, the reacting solution including hydrogen peroxide stored in the storage tank may be allowed to circulate between the storage tank and the reacting solution storage tank.

The apparatus may further include a reacting solution supply tank connected in communication with the reacting solution storage tank to supply the reacting solution to the reacting solution storage tank; and a hydrogen peroxide storage tank connected in communication with the storage tank to store the reacting solution including hydrogen peroxide stored in the storage tank.

The apparatus may further include a reacting solution transfer unit connecting the reacting solution supply tank, the reacting solution storage tank, the storage tank and the hydrogen peroxide storage tank to bring them into communication with each other so as to supply the reacting solution to each other, and the reacting solution transfer unit may include a pump; a first pipeline having an end connected to the reacting solution supply tank and an opposite end connected to the pump, and configured to supply the reacting solution stored in the reacting solution supply tank to the pump and shut off the supply as a first valve is open and closed; a second pipeline having an end connected to the pump and an opposite end connected to the reacting solution storage tank, and configured to supply the reacting solution having moved through the pump to the reacting solution storage tank and shut off the supply as a second valve is open and closed; a third pipeline having an end connected to the storage tank and an opposite end connected to the first pipeline, and configured to supply the reacting solution stored in the storage tank to the pump and shut off the supply as a third valve is open and closed; and a fourth pipeline having an end connected to the second pipeline and an opposite end connected to the hydrogen peroxide storage tank, and configured to supply the reacting solution stored in the storage tank to the hydrogen peroxide storage tank via the third pipeline, the pump and the second pipeline and shut off the supply as a fourth valve is open and closed.

### Advantageous Effects

Embodiments of the present disclosure may easily prepare the hydrogen peroxide solution only by electrostatic spraying of the reacting solution, for example, water.

Additionally, embodiments of the present disclosure may prepare the hydrogen peroxide solution only by electrostatic spraying of the reacting solution, for example, water, without expensive metal catalysts and high energy consumption, thereby achieving cost savings.

Furthermore, embodiments of the present disclosure may supply the reacting solution including hydrogen peroxide produced through electrostatic spraying to desired locations to be used in disinfection, sterilization or cleaning applications.

Further, embodiments of the present disclosure may easily produce the desired concentration of hydrogen peroxide by circulating the reacting solution including hydrogen peroxide so that the concentration of hydrogen peroxide produced reaches the preset value.

### DESCRIPTION OF DRAWINGS

FIG. 1 is a conceptual diagram of an apparatus for preparing a hydrogen peroxide solution using electrostatic spraying according to a first embodiment of the present disclosure.

FIG. 2 is a diagram showing the operation of an apparatus for preparing a hydrogen peroxide solution using electrostatic spraying according to a first embodiment of the present disclosure.

FIG. 3 is a diagram showing a relative position of a nozzle tip with respect to a nozzle body according to a first embodiment of the present disclosure.

FIG. 4 is a diagram showing a relative position of a ground unit with respect to a nozzle unit according to a first embodiment of the present disclosure.

FIG. 5 is a perspective view showing an apparatus for preparing a hydrogen peroxide solution using electrostatic spraying according to a second embodiment of the present disclosure.

FIG. 6 is an exploded perspective view showing an apparatus for preparing a hydrogen peroxide solution using electrostatic spraying according to a second embodiment of the present disclosure.

FIG. 7 is a side view showing an apparatus for preparing a hydrogen peroxide solution using electrostatic spraying according to a second embodiment of the present disclosure.

FIG. 8 is a diagram showing an operation of supplying a reacting solution from a reacting solution supply tank to a reacting solution storage tank in an apparatus for preparing a hydrogen peroxide solution using electrostatic spraying according to a second embodiment of the present disclosure.

FIG. 9 is a diagram showing an operation of circulating a reacting solution in a reacting solution storage tank and a storage tank in an apparatus for preparing a hydrogen peroxide solution using electrostatic spraying according to a second embodiment of the present disclosure.

FIG. 10 is a diagram showing an operation of supplying a reacting solution stored in a storage tank to a hydrogen peroxide storage tank in an apparatus for preparing a hydrogen peroxide solution using electrostatic spraying according to a second embodiment of the present disclosure.

### BEST MODE

To sufficiently understand the present disclosure, operational advantages of the present disclosure and objectives achieved by the practice of the present disclosure, reference is made to the accompanying drawings illustrating exemplary embodiments of the present disclosure and the disclosure in the accompanying drawings.

Hereinafter, the present disclosure will be described in detail by describing the exemplary embodiments of the present disclosure with reference to the accompanying drawings. In each drawing, the same reference numeral indicates the same element.

Hereinafter, an apparatus for preparing a hydrogen peroxide solution using electrostatic spraying according to a first embodiment of the present disclosure will be described.

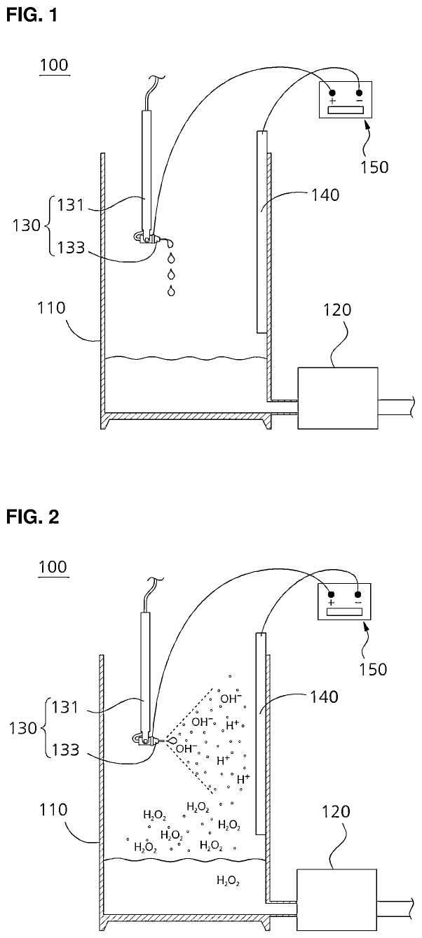

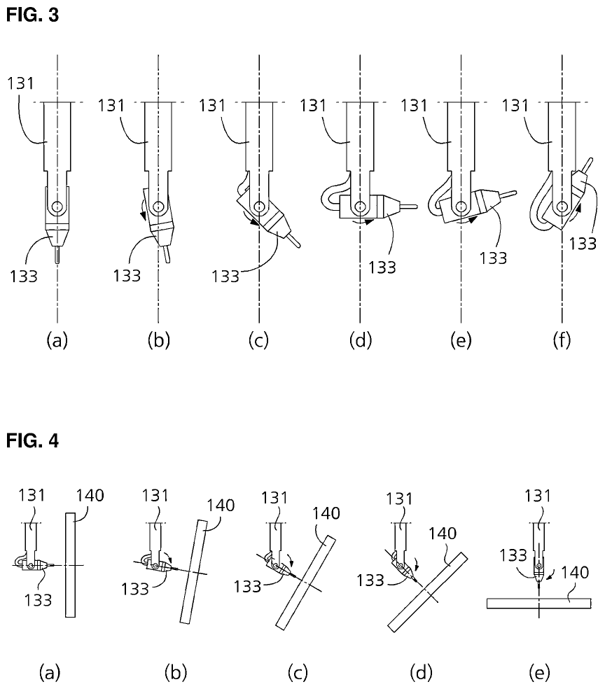

FIG. 1 is a conceptual diagram of the apparatus for preparing the hydrogen peroxide solution using electrostatic spraying according to the first embodiment of the present disclosure, FIG. 2 is a diagram showing the operation of the apparatus for preparing the hydrogen peroxide solution using electrostatic spraying according to the first embodiment of the present disclosure, FIG. 3 is a diagram showing a relative position of a nozzle tip with respect to a nozzle body according to the first embodiment of the present disclosure, and FIG. 4 is a diagram showing a relative position of a ground unit with respect to a nozzle unit according to the first embodiment of the present disclosure.

Referring to FIGS. 1 to 4, the apparatus 100 for preparing the hydrogen peroxide solution using electrostatic spraying according to the first embodiment of the present disclosure includes the nozzle unit 130 to spray a reacting solution, the ground unit 140 disposed opposite the nozzle unit 130, a power supply unit 150 to apply a high voltage between the nozzle unit 130 and the ground unit 140, a storage tank 110 disposed below the nozzle unit 130 and the ground unit 140, and a filter unit 120 connected in communication with the storage tank 110.

In this embodiment, the reacting solution may include distilled water, spring water, mineral water, deep sea water, ionized water, etc.

Additionally, the reacting solution may include 1 to 200 mg/L of minerals. The minerals may include at least one of iron, magnesium, potassium, calcium, sodium, silicon or phosphorus. Additionally, a pH of the reacting solution may be from 5 to 10, and preferably from 6.7 to 7.3.

Additionally, in the reacting solution, evaporation residue may be from 30 to 200 mg/L, free carbon dioxide may be from 3 to 30 mg/L, Threshold Odor Number (TON) may be 3 or less, water hardness may be from 10 to 100 mg/L, potassium permanganate consumption may be 3 mg/L or less, and residual chlorine may be 0.4 mg/L or less.

The evaporation residue is the residue after evaporation of water, and indicates the amount of minerals or organic matter floating or dissolved in water. The free carbon dioxide is carbon dioxide gas dissolved in water and may provide effervescence and refreshing taste when it is added in large amounts. The potassium permanganate consumption indicates the amount of organic matter, for example, carbon compounds, beyond the ability of microorganisms to decompose organic matter to purify water. The residual chlorine indicates the amount of chlorine used for disinfection, and when the concentration is high, it may give an unpleasant taste. Chlorine disinfection used to purify water all over the world is performed to ensure safety from microorganisms such as bacteria, colon bacillus, etc., before transportation from water purification plant to home, and the Korean Law states “residual chlorine at the faucet is maintained at 0.1 mg/L or more”. Chlorine is considered as an essential measure for ensuring safety of tap water. The chlorine disinfection results in by-products such as chloroform, trihalomethane, etc., and these materials have cancer risks and are strictly regulated by the legal standards governing water quality. The water hardness indicates the amounts of calcium and magnesium.

This embodiment relates to hydrogen peroxide production using electrostatic spraying. To this end, this embodiment includes the nozzle unit 130 to spray the reacting solution, the ground unit 140 disposed opposite the nozzle unit 130 and the power supply unit 150 to apply a high voltage between the nozzle unit 130 and the ground unit 140.

The nozzle unit 130 includes the nozzle body 131 to supply the reacting solution, and the nozzle tip 133 connected to the nozzle body 131, disposed opposite the ground unit 140 and having an outlet through which the reacting solution is sprayed.

Additionally, as shown in FIG. 3, the nozzle tip 133 may be rotatably connected to the nozzle body 131. Accordingly, the nozzle tip 133 may be disposed in a coaxial direction or inclined with respect to the nozzle body. As shown in FIG. 4, this is to rotate the nozzle tip 133 relative to the nozzle body 131 according to the location of the ground unit 140 so that the outlet direction of the nozzle tip 133 faces the ground unit 140.

Additionally, as shown in FIG. 1, the average water droplet size of the reacting solution coming out of the outlet of the nozzle tip 133 may be from 10 nm to 200 μm. This is to atomize the reacting solution sprayed from the nozzle tip 133 into fine droplets having the droplet size of 20 μm or less by electrostatic spraying.

The diameter of the outlet is 4 mm or less so that the average water droplet size of the reacting solution coming out of the outlet is from 10 nm to 200 μm. When the diameter of the outlet is larger than 4 mm, the average water droplet size may be larger than 200 μm, and when the average water droplet size is larger than 200 μm, the concentration of hydrogen peroxide produced may be lower.

For the electrostatic spraying effect, a positive (+) voltage may be applied to the nozzle tip 133 for spraying the reacting solution, and a high negative (−) voltage may be applied to the ground unit 140 spaced a predetermined distance apart from the nozzle tip 133.

The electrostatic spraying refers to a phenomenon in which as ions in the nozzle tip 133 and the reacting solution move to the reacting solution surface by attraction and repulsion, when the Coulomb repulsion force is greater than the surface tension of water droplets of the reacting solution, water droplets of the reacting solution are atomized and sprayed.

When the voltage between the nozzle tip 133 and the ground unit 140 is low, the electric force acting on the surface of water droplets of the reacting solution and the repulsion force of cations is smaller than the surface tension of water droplets of the reacting solution and water droplets of the reacting solution are not sprayed, but when high voltage is applied between the nozzle tip 133 and the ground unit 140, the electric force acting on the surface of water droplets of the reacting solution and the repulsion force of cations is greater than the surface tension of water droplets of the reacting solution and water droplets of the reacting solution are atomized and sprayed from the nozzle tip 133.

In this instance, when the average water droplet size of the reacting solution is below a submicron level, some of water droplets of the reacting solution break apart as shown in the following [Chemical Formula 1], and at the same time, produce hydrogen peroxide as shown in the following [Chemical Formula 2]. That is, water droplets of the reacting solution may be atomized by electrostatic spraying and hydrogen ions (H⁺) may react with hydroxide ions (OH⁻) again to produce hydrogen peroxide.

H₂O—>H⁺+OH⁻ [Chemical Formula 1]

O₂+2H⁺+2e⁻->H₂O₂,2OH⁻+2H⁺—>H₂O₂+H₂

This embodiment may effectively produce hydrogen peroxide with only the reacting solution and oxygen in air by the electrostatic spraying effect. Accordingly, it may be possible to prepare the hydrogen peroxide solution without additional chemicals such as flammable hydrogen gas or oxygen gas that supports combustion, expensive metal catalysts and high energy consumption, thereby achieving cost savings.

Additionally, according to this embodiment, when producing hydrogen peroxide using electrostatic spraying, there is no explosion risk such as heat generation, the process is simple and straightforward, and it is possible to produce hydrogen peroxide at a small scale as well as a large scale, and achieve on-site production and use of hydrogen peroxide, thereby reducing the transportation and storage costs of hydrogen peroxide.

Additionally, in this embodiment, when hydrogen peroxide is produced using electrostatic spraying, hydrogen peroxide may be stored together with the reacting solution such as water and the stored reacting solution including hydrogen peroxide may be supplied to desired locations to be used in disinfection, sterilization or cleaning applications.

In this embodiment, the production volume of hydrogen peroxide solution in the reacting solution is from 0.001 mL/min to 0.05 mL/min per supply volume (mL/min) of the reacting solution coming out of the nozzle tip 133.

To this end, the outlet pressure of the reacting solution coming out of the outlet of the nozzle tip 133 is higher than 0 bar and equal to and lower than 10 bar. When the outlet pressure of the reacting solution is higher than 10 bar, shorts may occur. Additionally, the supply volume of the reacting solution coming out of the nozzle tip 133 is 0.01 mL/min or more. The supply volume of less than 0.01 mL/min may result in low production volume of hydrogen peroxide.

Additionally, to prevent shorts between the outlet of the nozzle tip 133 and the ground unit 140 during electrostatic spraying of the reacting solution, the gap between the outlet of the nozzle tip 133 and the ground unit 140 may be 10 mm or more. Additionally, the applied voltage between the nozzle tip 133 and the ground unit 140 may be±5 kV. When the gap between the outlet of the nozzle tip 133 and the ground unit 140 is less than 10 mm, and the applied voltage is less than ±5 kV, the concentration of hydrogen peroxide produced may be lower.

Meanwhile, the reacting solution including hydrogen peroxide produced through electrostatic spraying is stored in the storage tank 110. The storage tank 110 may be disposed below the nozzle unit 130 and the ground unit 140. In this instance, the nozzle unit 130 may be disposed at the upper inner part of the storage tank 110 and the ground unit 140 may be vertically fixed to the inner wall of the storage tank 110 (see FIG. 1). Additionally, as shown in FIG. 4, when the ground unit 140 is disposed at an angle or horizontally inside the storage tank 110, the nozzle tip 133 may be disposed opposite the ground unit 140 by the relative rotation with respect to the nozzle body 131.

Additionally, in this embodiment, to remove chlorine from the reacting solution including hydrogen peroxide stored in the storage tank 110, the filter unit 120 is connected in communication with the storage tank 110. In this embodiment, the filter unit 120 may include a carbon filter to remove chlorine from the reacting solution.

The reacting solution including hydrogen peroxide and chlorine stored in the storage tank 110 may undergo the chlorine removal through the filter unit 120 and then may be stored in a container for transportation or transported to a desired location.

Hereinafter, an apparatus for preparing a hydrogen peroxide solution using electrostatic spraying according to a second embodiment of the present disclosure will be described.

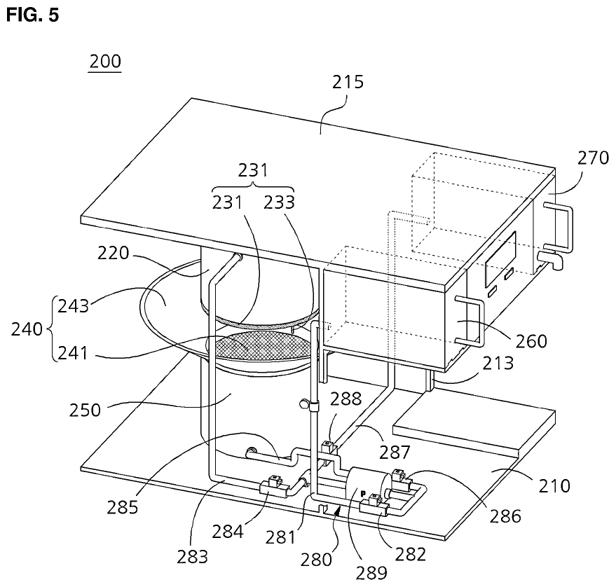

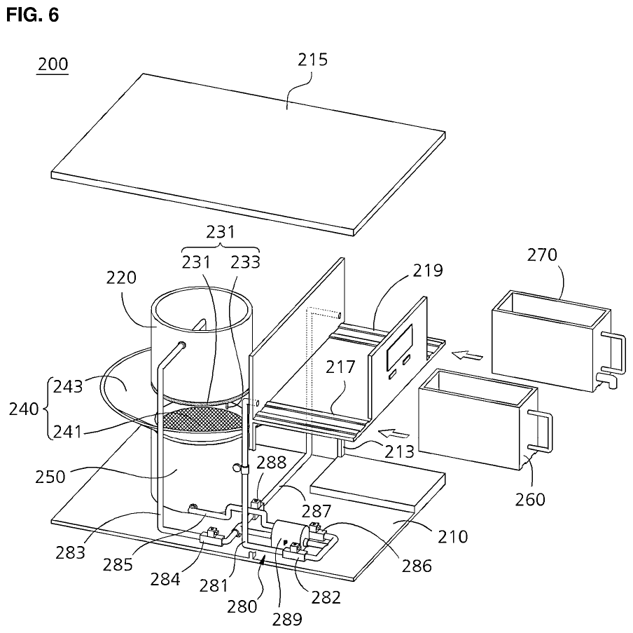

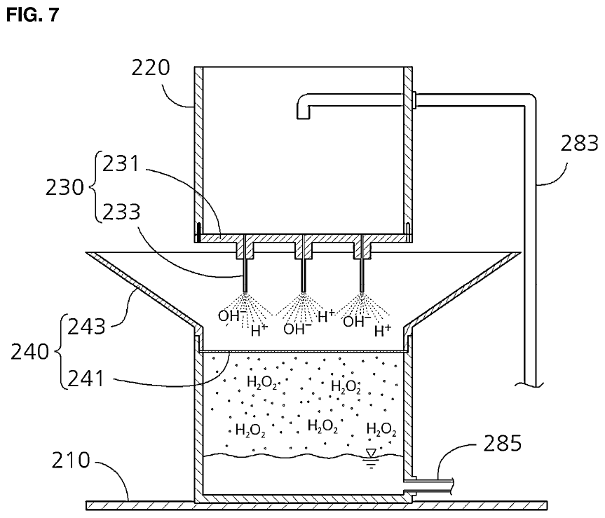

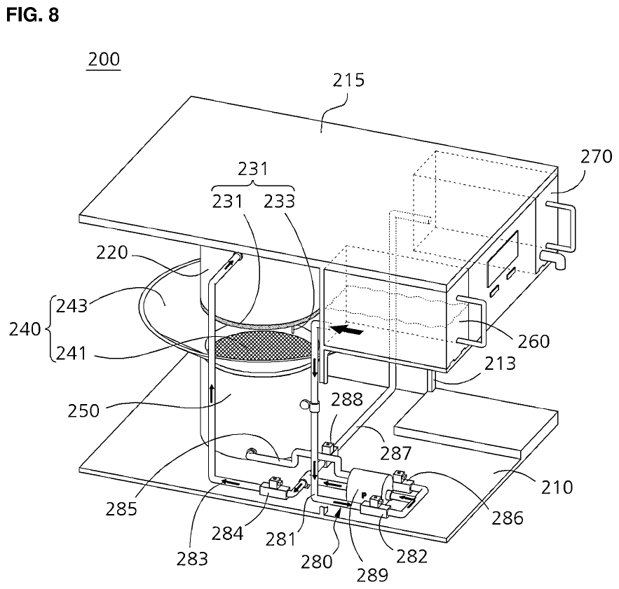

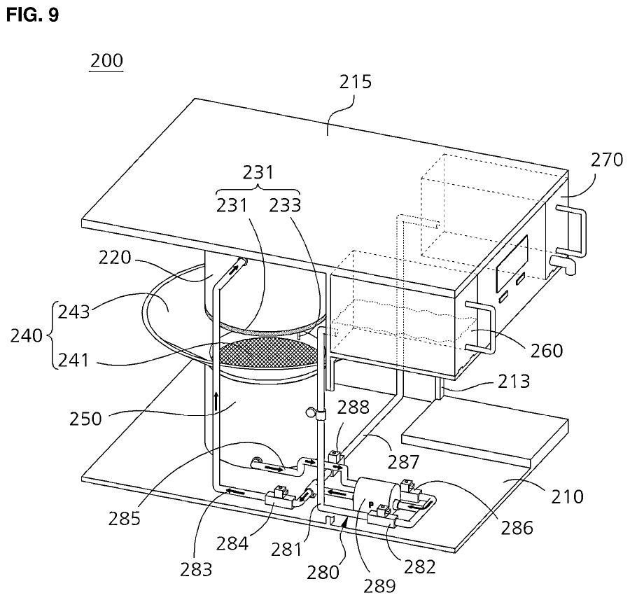

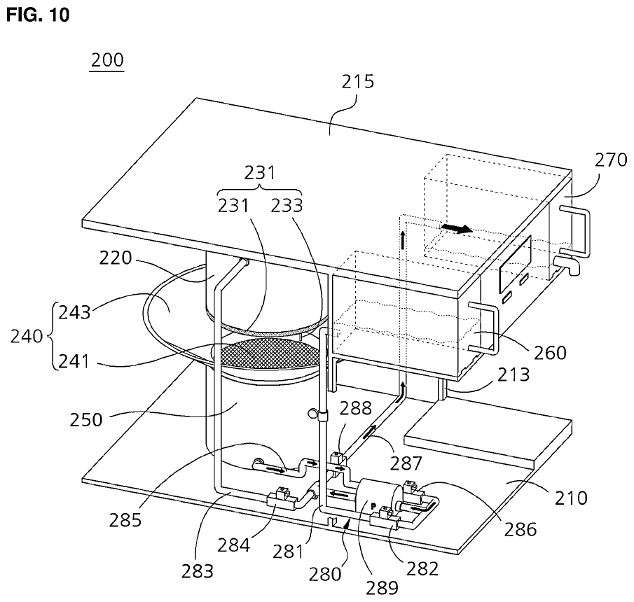

FIG. 5 is a perspective view showing the apparatus for preparing the hydrogen peroxide solution using electrostatic spraying according to the second embodiment of the present disclosure, FIG. 6 is an exploded perspective view showing the apparatus for preparing the hydrogen peroxide solution using electrostatic spraying according to the second embodiment of the present disclosure, FIG. 7 is a side view showing the apparatus for preparing the hydrogen peroxide solution using electrostatic spraying according to the second embodiment of the present disclosure, FIG. 8 is a diagram showing the operation of supplying the reacting solution from a reacting solution supply tank to a reacting solution storage tank in the apparatus for preparing the hydrogen peroxide solution using electrostatic spraying according to the second embodiment of the present disclosure, FIG. 9 is a diagram showing the operation of circulating the reacting solution in the reacting solution storage tank and a storage tank in the apparatus for preparing the hydrogen peroxide solution using electrostatic spraying according to the second embodiment of the present disclosure, and FIG. 10 is a diagram showing the operation of supplying the reacting solution stored in the storage tank to a hydrogen peroxide storage tank in the apparatus for preparing the hydrogen peroxide solution using electrostatic spraying according to the second embodiment of the present disclosure.

Referring to FIGS. 5 to 10, the apparatus 200 for preparing the hydrogen peroxide solution using electrostatic spraying according to the second embodiment of the present disclosure includes the reacting solution storage tank 220 to store the reacting solution, a needle unit 230 disposed below the reacting solution storage tank 220 to spray the reacting solution, a ground unit 240 disposed opposite the needle unit 230, the storage tank 250 disposed below the ground unit 240 to store the reacting solution including hydrogen peroxide, a power supply unit (not shown) to apply a high voltage between the needle unit 230 and the ground unit 240, the reacting solution supply tank 260 connected in communication with the reacting solution storage tank 220 to supply the reacting solution to the reacting solution storage tank 220, the hydrogen peroxide storage tank 270 connected in communication with the storage tank 250 to store the reacting solution including hydrogen peroxide stored in the storage tank 250, and a reacting solution transfer unit 280 connecting the reacting solution supply tank 260, the reacting solution storage tank 220, the storage tank 250 and the hydrogen peroxide storage tank 270 to bring them into communication with each other so as to supply the reacting solution to each other.

The description of the reacting solution and the electrostatic spraying in this embodiment is the same as the above description of the first embodiment of the present disclosure and its detailed description is omitted.

The reacting solution storage tank 220 stores the reacting solution supplied from the reacting solution supply tank 260. The reacting solution storage tank 220 is formed as a non-conductor. Additionally, the reacting solution storage tank 220 is fixedly installed at the lower surface of an upper frame 215.

The needle unit 230 is disposed below the reacting solution storage tank 220, and the reacting solution stored in the reacting solution storage tank 220 drops down through the needle unit 230 by the differential head.

The needle unit 230 includes a conductive plate 231 coupled to the lower surface of the reacting solution storage tank 220, and at least one needle 233 coupled to the lower surface of the conductive plate 231 and disposed opposite the ground unit 240 to spray the reacting solution. One needle 233 may be present at the center of the conductive plate 231, or a plurality of needles 233 may be present in the conductive plate 231, spaced apart from each other.

The average water droplet size of the reacting solution sprayed from the needle 233 may be from 10 nm to 200 μm. This is to atomize the reacting solution sprayed from the needle 233 into fine droplets having the droplet size of 20 μm or less by electrostatic spraying.

Additionally, as described above, the ground unit 240 may be disposed below the at least one needle 233, spaced a predetermined distance apart from the needle 233.

The ground unit 240 includes a mesh-type ground plate 241 disposed below the needle unit 230, and a cone-shaped anti-leak plate 243 coupled to the edge of the ground plate 241 and inclined upward.

The ground plate 241 is of mesh type, and the reacting solution electrostatic sprayed from the needle 233 drops down through the mesh-type ground plate 241.

The anti-leak plate 243 prevents leaks of the reacting solution electrostatic sprayed from the at least one needle 233. Accordingly, the anti-leak plate 243 is disposed around the needle 233 such that the upper end is spaced a predetermined distance apart from the at least one needle 233. Additionally, the anti-leak plate 243 has the cone shape, which allows the reacting solution electrostatic sprayed from the needle 233 to flow down by its weight.

Additionally, to produce hydrogen peroxide from the reacting solution sprayed through the needle 233 by electrostatic spraying, the power supply unit applies a positive (+) voltage to the needle 233 for spraying the reacting solution and a negative (−) voltage to the ground plate 241 spaced the predetermined distance apart from the needle 233.

In this embodiment, the production volume of the hydrogen peroxide solution in the reacting solution is from 0.001 mL/min to 0.05 mL/min per supply volume (mL/min) of the reacting solution sprayed from the needle 233.

To this end, the spray pressure of the reacting solution sprayed from the needle 233 is higher than 0 bar and equal to and lower than 10 bar. Additionally, the gap between the tip of the needle 233 and the ground plate 241 may be 10 mm or more to prevent shorts between the tip of the needle 233 and the ground plate 241 during electrostatic spraying of the reacting solution. Additionally, the applied voltage between the needle 233 and the ground plate 241 may be±5 kV.

Additionally, as shown in FIG. 7, when the high voltage is applied between the plurality of needles 233 and the ground plate 241, water droplets sprayed from the plurality of needles 233 may be atomized by electrostatic spraying and hydrogen ions (H⁺) may react with hydroxide ions (OH⁻) again to produce hydrogen peroxide.

Additionally, the hydrogen peroxide and the reacting solution including hydrogen peroxide drop down through the mesh-type ground plate 241 or along the anti-leak plate 243 and are stored in the storage tank 250 below the ground plate 241.

The storage tank 250 may be fixedly installed on the upper surface of a lower frame 210. In this embodiment, the ground plate 241 may be fixedly installed on top of the storage tank 250, and the anti-leak plate 243 may be fixedly installed at the upper edge of the storage tank 250 (see FIG. 5). Accordingly, the electrostatic sprayed reacting solution may be stored in the storage tank 250 through the ground plate 241 or along the anti-leak plate 243 without leaks.

Additionally, the reacting solution including hydrogen peroxide stored in the storage tank 250 may be supplied to the hydrogen peroxide storage tank 270.

Meanwhile, in this embodiment, the reacting solution supply tank 260 and the hydrogen peroxide storage tank 270 may be detachably coupled to a first receiving portion 217 and a second receiving portion 219 of the upper frame 215, respectively (see FIG. 6). The upper frame 215 is supported by a side frame 213 fixedly installed at the lower frame 210.

Additionally, the movement of the reacting solution described above is made by the reacting solution transfer unit 280, and the reacting solution transfer unit 280 connects the reacting solution supply tank 260, the reacting solution storage tank 220, the storage tank 250 and the hydrogen peroxide storage tank 270 to bring them into communication with each other so as to supply the reacting solution to each other.

Specifically, the reacting solution transfer unit 280 includes a pump 289, a first pipeline 281 having an end connected to the reacting solution supply tank 260 and an opposite end connected to the pump 289, a second pipeline 283 having an end connected to the pump 289 and an opposite end connected to the reacting solution storage tank 220, a third pipeline 285 having an end connected to the storage tank 250 and an opposite end connected to the first pipeline 281, and a fourth pipeline 287 having an end connected to the second pipeline 283 and an opposite end connected to the hydrogen peroxide storage tank 270.

Additionally, a first valve 282 is installed at the first pipeline 281, and as the first valve 282 is open and closed, the reacting solution stored in the reacting solution supply tank 260 is supplied to the pump 289 and the supply is shut off. Additionally, a second valve 284 is installed at the second pipeline 283, and as the second valve 284 is open and closed, the reacting solution having moved through the pump 289 is supplied to the reacting solution storage tank 220 and the supply is shut off. Additionally, a third valve 286 is installed at the third pipeline 285, and as the third valve 286 is open and closed, the reacting solution stored in the storage tank 250 is supplied to the pump 289 and the supply is shut off. Additionally, a fourth valve 288 is installed at the fourth pipeline 287, and as the fourth valve 288 is open and closed, the reacting solution stored in the storage tank 250 is supplied to the hydrogen peroxide storage tank 270 via the third pipeline 285, the pump 289 and the second pipeline 283 and the supply is shut off. The first to fourth valves 288 according to this embodiment may be a solenoid valve, but the scope of protection of the present disclosure is not limited thereby.

Describing the operation of supplying the reacting solution from the reacting solution supply tank 260 to the reacting solution storage tank 220, as shown in FIG. 8, the first valve 282 and the second valve 284 are open and the third valve 286 and the fourth valve 288 are closed. Additionally, by the operation of the pump 289, the reacting solution stored in the reacting solution supply tank 260 is supplied to the reacting solution storage tank 220 through the first pipeline 281 and the second pipeline 283.

Additionally, the reacting solution stored in the reacting solution storage tank 220 is atomized and electrostatic sprayed through the at least one needle 233 and hydrogen ions react with hydroxide ions again to produce hydrogen peroxide. Additionally, the remaining reacting solution and the produced hydrogen peroxide drop down and are stored in the storage tank 250.

In this instance, when the concentration (ppm) of hydrogen peroxide stored in the storage tank 250 is less than a preset value, in this embodiment, the reacting solution including hydrogen peroxide stored in the storage tank 250 may be allowed to circulate between the storage tank 250 and the reacting solution storage tank 220 so that the concentration of hydrogen peroxide reaches the preset value.

Describing the operation of circulating the reacting solution including hydrogen peroxide between the storage tank 250 and the reacting solution storage tank 220, as shown in FIG. 9, the second valve 284 and the third valve 286 are open and the first valve 282 and the fourth valve 288 are closed. Additionally, by the operation of the pump 289, the reacting solution including hydrogen peroxide stored in the storage tank 250 is supplied to the reacting solution storage tank 220 via the third pipeline 285 and the second pipeline 283. Additionally, the above-described operation repeats until the concentration of hydrogen peroxide stored in the storage tank 250 reaches the preset value.

Meanwhile, when the concentration of hydrogen peroxide reaches the preset value, the reacting solution including hydrogen peroxide stored in the storage tank 250 is supplied to the hydrogen peroxide storage tank 270.

Describing the operation of supplying the reacting solution including hydrogen peroxide from the storage tank 250 to the hydrogen peroxide storage tank 270, as shown in FIG. 10, the third valve 286 and the fourth valve 288 are open and the first valve 282 and the second valve 284 are closed. Additionally, by the operation of the pump 289, the reacting solution stored in the storage tank 250 is supplied to the hydrogen peroxide storage tank 270 via the third pipeline 285 and the fourth pipeline 287.

Although not shown, to remove chlorine from the reacting solution including hydrogen peroxide stored in the hydrogen peroxide storage tank 270, a filter unit (not shown) may be connected in communication with the hydrogen peroxide storage tank 270. In this embodiment, the filter unit may include a carbon filter to remove chlorine from the reacting solution. The reacting solution including hydrogen peroxide and chlorine stored in the hydrogen peroxide storage tank 270 may undergo the chlorine removal through the filter unit and then may be stored in a container for transportation or transported to a desired location.

The present disclosure is not limited to the disclosed embodiments, and it is obvious to persons having ordinary skill in the technical field that a variety of modifications and changes may be made thereto without departing from the spirit and scope of the present disclosure. Accordingly, such modifications or variations fall within the scope of protection of the present disclosure.

[Detailed Description of Main Elements]

| | |
|---|---|
| 100: Apparatus for preparing | |
| a hydrogen peroxide | |
| solution using | |
| electrostatic spraying | |
| 110: Storage tank | |
| 120: Filter unit | 130: Nozzle unit |
| 131: Nozzle body | 133: Nozzle tip |
| 140: Ground unit | 150: Power supply unit |
| 200: Apparatus for preparing | |
| a hydrogen peroxide solution | |
| using electrostatic spraying | |
| 210: Lower frame | |
| 213: Side frame | 215: Upper frame |
| 217: First receiving portion | 219: Second receiving portion |
| 220: Reacting solution storage tank | 230: Needle unit |
| 231: Conductive plate | 233: Needle |
| 240: Ground unit | 241: Ground plate |
| 243: Anti-leak plate | 250: Storage tank |
| 260: Reacting solution supply tank | |
| 270: Hydrogen peroxide storage tank | |
| 280: Reacting solution transfer unit | 281: First pipeline |
| 282: First valve | 283: Second pipeline |
| 284: Second valve | 285: Third pipeline |
| 286: Third valve | 287: Fourth pipeline |
| 288: Fourth valve | 289: Pump |

### INDUSTRIAL APPLICABILITY

The present disclosure may produce hydrogen peroxide without expensive metal catalysts and high energy consumption by atomizing the reacting solution into fine droplets through electrostatic spraying and reacting hydrogen ions with hydroxide ions again.

The invention claimed is:

1. An apparatus for preparing a hydrogen peroxide solution using electrostatic spraying, the apparatus comprising:
a nozzle unit configured to spray a reacting solution;
a ground unit disposed opposite the nozzle unit; and
a power supply unit configured to apply a high voltage between the nozzle unit and the ground unit,
wherein the reacting solution is atomized into fine droplets having a droplet size of 20 μm or less and electrostatic sprayed from the nozzle unit and hydrogen ions (H⁺) react with hydroxide ions (OH⁻) again to produce hydrogen peroxide.

2. The apparatus for preparing the hydrogen peroxide solution using electrostatic spraying according to claim 1, wherein the nozzle unit includes:
a nozzle body configured to supply the reacting solution; and
a nozzle tip connected to the nozzle body, disposed opposite the ground unit and having an outlet through which the reacting solution is sprayed, and
wherein an average water droplet size of the reacting solution coming out of the outlet is from 10 nm to 200 μm.

3. The apparatus for preparing the hydrogen peroxide solution using electrostatic spraying according to claim 2, wherein the nozzle tip is rotatably connected to the nozzle body such that the nozzle tip is disposed in a coaxial direction or inclined with respect to the nozzle body.

4. An apparatus for preparing a hydrogen peroxide solution using electrostatic spraying, further comprising:
a storage tank disposed below the nozzle unit and the ground unit to store the reacting solution including hydrogen peroxide produced through electrostatic spraying; and
a filter unit connected in communication with the storage tank and configured to remove chlorine from the reacting solution including hydrogen peroxide.

5. An apparatus for preparing a hydrogen peroxide solution using electrostatic spraying, the apparatus comprising:
a reacting solution storage tank configured to store a reacting solution;
a needle unit disposed below the reacting solution storage tank, and configured to spray the reacting solution fed from the reacting solution storage tank;
a ground unit located below the needle unit and disposed opposite the needle unit;
a storage tank disposed below the ground unit and configured to store the reacting solution including hydrogen peroxide; and
a power supply unit configured to apply a high voltage between the nozzle unit and the ground unit,
wherein the reacting solution is atomized into fine droplets having a droplet size of 20 μm or less and electrostatic sprayed from the needle unit and hydrogen ions (H⁺) react with hydroxide ions (OH⁻) again to produce hydrogen peroxide.

6. The apparatus for preparing the hydrogen peroxide solution using electrostatic spraying according to claim 5, wherein the ground unit includes:
a mesh-type ground plate disposed below the needle unit; and
a cone-shaped anti-leak plate coupled to an edge of the ground plate and inclined upward.

7. The apparatus for preparing the hydrogen peroxide solution using electrostatic spraying according to claim 5, wherein the needle unit includes:
a conductive plate coupled to a lower surface of the reacting solution storage tank; and
at least one needle coupled to a lower surface of the conductive plate and disposed opposite the ground unit to spray the reacting solution.

8. The apparatus for preparing the hydrogen peroxide solution using electrostatic spraying according to claim 5, wherein when a concentration (ppm) of hydrogen peroxide stored in the storage tank is less than a preset value, the reacting solution including hydrogen peroxide stored in the storage tank is allowed to circulate between the storage tank and the reacting solution storage tank.

9. The apparatus for preparing the hydrogen peroxide solution using electrostatic spraying according to claim 5, further comprising:
a reacting solution supply tank connected in communication with the reacting solution storage tank to supply the reacting solution to the reacting solution storage tank; and
a hydrogen peroxide storage tank connected in communication with the storage tank to store the reacting solution including hydrogen peroxide stored in the storage tank.

10. The apparatus for preparing the hydrogen peroxide solution using electrostatic spraying according to claim 9, further comprising:
a reacting solution transfer unit connecting the reacting solution supply tank, the reacting solution storage tank, the storage tank and the hydrogen peroxide storage tank to bring them into communication with each other so as to supply the reacting solution to each other,
wherein the reacting solution transfer unit includes:
a pump;
a first pipeline having an end connected to the reacting solution supply tank and an opposite end connected to the pump, and configured to supply the reacting solution stored in the reacting solution supply tank to the pump and shut off the supply as a first valve is open and closed;
a second pipeline having an end connected to the pump and an opposite end connected to the reacting solution storage tank, and configured to supply the reacting solution having moved through the pump to the reacting solution storage tank and shut off the supply as a second valve is open and closed;
a third pipeline having an end connected to the storage tank and an opposite end connected to the first pipeline, and configured to supply the reacting solution stored in the storage tank to the pump and shut off the supply as a third valve is open and closed; and
a fourth pipeline having an end connected to the second pipeline and an opposite end connected to the hydrogen peroxide storage tank, and configured to supply the reacting solution stored in the storage tank to the hydrogen peroxide storage tank via the third pipeline, the pump and the second pipeline and shut off the supply as a fourth valve is open and closed.
